(19) United States
(12) Patent Application Publication
(10) Pub. No.: US 2026/0354985 A1
(43) Pub. Date: Nov. 12, 2026

(54) INTERVERTEBRAL IMPLANT FOR SPACING CERVICAL VERTEBRAE APART, IMPLANTATION SYSTEM, AND USE THEREOF

(71) Applicants: Beijing Naton Medical Technology Holdings Co., Ltd., Beijing (CN); Tianjin Zhengtian Medical Instrument Co.,Ltd, Tianjin (CN)

(72) Inventors: Peng Xu, Beijing (CN); Guoqiang Ma, Beijing (CN); Xun Zhang, Beijing (CN); Xiang Dong, Beijing (CN); Hongping Duan, Beijing (CN); Shufu Xu, Beijing (CN)

(73) Assignees: Beijing Naton Medical Technology Holdings Co., Ltd., Beijing (CN); Tianjin Zhengtian Medical Instrument Co.,Ltd, Tianjin (CN)

(21) Appl. No.: 19/139,264

(22) PCT Filed: Nov. 24, 2023
(86) PCT No.: PCT/CN2023/134031

§ 371 Date: Jun. 13, 2025

(30) Foreign Application Priority Data
Dec. 16, 2022 (CN) 202211637094.4

(51) Int. Cl. A61F 2/44; A61B 17/68; A61B 17/70; A61F 2/30; A61F 2/46

(52) U.S. Cl. CPC A61F 2/447; A61B 17/70; A61F 2/30734; A61F 2/30749; A61F 2/30771; A61F 2/4611; A61B 2017/681; A61F 2002/30112; A61F 2002/30372; A61F 2002/30556; A61F 2002/30622; A61F 2002/30738; A61F 2002/30843; A61F 2002/3093

(57) ABSTRACT

An intervertebral implant for distracting a cervical vertebrae includes: a first support body and a second support body, the first support body and the second support body being separately provided along a height direction of the intervertebral implant to form a bone accommodation cavity, the first support body being provided with a first inclined slot that is inclined relative to the height direction of the intervertebral implant, and the first inclined slot being provided with a notch open towards the second support body; and a driving component, provided between the first support body and the second support body in the height direction of the intervertebral implant and including a pushing component and a first sliding column connected with each other.

### CROSS-REFERENCE TO RELATED APPLICATIONS

This application is the U.S. National Stage Application of International Application No. PCT/CN2023/134031, which claims the priority to Chinese Patent Application No. 2022116370944, filed with the State Intellectual Property Office of P. R. China on Dec. 16, 2022, the entire contents of which are incorporated herein by reference.

### TECHNICAL FIELD

The disclosure relates to a technical field of medical devices, and particularly to an intervertebral implant for distracting a cervical vertebrae, an implant system including the intervertebral implant, and an application.

### BACKGROUND

An intervertebral fusion technology of a cervical vertebrae may effectively treat a problem such as degeneration and instability of the cervical vertebrae. The intervertebral fusion technology may fuse upper and lower vertebral bodies, which has an effect of maintaining a height of an intervertebral space, restoring a physiological anterior convexity, relieving a pressure on a nerve root, and maintaining a spinal stability, with a satisfactory clinical result, a minimal surgical trauma, and an operation convenience. In the related art, an intervertebral implant includes a first support body, a second support body, and an intermediate body with a wedge-shaped surface provided between the first support body and the second support body. The first support body and the second support body are pushed to be close to or farther away from each other by the wedge-shaped surface of the intermediate body, to realize adjustment of a height of the intervertebral implant. However, a structure of the intermediate body occupies a space of a bone accommodation cavity, resulting in a problem of a small bone implant cavity, thus resulting in a problem of a poor stability and a fusion performance of the intervertebral implant.

### SUMMARY

A first aspect of embodiments of the disclosure provides an intervertebral implant for distracting a cervical vertebrae. The intervertebral implant for distracting the cervical vertebrae in embodiments of the disclosure includes a driving component, a first support body, and a second support body. The intervertebral implant has an expanded state and a collapsed state. The first support body and the second support body is separately provided along a height direction of the intervertebral implant to form a bone accommodation cavity, the first support body is provided with a first inclined slot that is inclined relative to the height direction of the intervertebral implant, and the first inclined slot is provided with a notch open towards the second support body. The driving component is provided between the first support body and the second support body in the height direction of the intervertebral implant, the driving component includes a pushing component and a first sliding column connected with each other, the first sliding column cooperatively slides in the first inclined slot, and the pushing component moves along the height direction of the intervertebral implant and perpendicular to an extending direction of the first sliding column, to realize transition between the collapsed state and the expanded state of the intervertebral implant by the pushing component driving the first sliding column to move along an inclined direction of the first inclined slot.

A second aspect of embodiments of the disclosure further provides an intervertebral implantation system. The intervertebral implantation system includes an implanter and the intervertebral implant for distracting the cervical vertebrae. The implanter is connected to the pushing component and configured to adjust a position of the pushing component, to realize the transition between the collapsed state and the expanded state of the intervertebral implant by the pushing component driving the first sliding column to move along the inclined direction of the first inclined slot.

A third aspect of embodiments of the disclosure provides an application of the intervertebral implant for distracting the cervical vertebrae. The intervertebral implant is applied to the cervical vertebra, a thoracic vertebra, or a lumbar vertebra.

### BRIEF DESCRIPTION OF THE DRAWINGS

FIG. 1 is a three-dimensional diagram illustrating an intervertebral implant for distracting a cervical vertebrae according to an embodiment of the disclosure.

FIG. 2 is a three-dimensional diagram illustrating an intervertebral implant for distracting a cervical vertebrae, omitting a first support body, according to an embodiment of the disclosure.

FIG. 3 is a three-dimensional diagram illustrating an intervertebral implant for distracting a cervical vertebrae, omitting a positioning frame, according to an embodiment of the disclosure.

FIG. 4 is a three-dimensional diagram illustrating an intervertebral implant for distracting a cervical vertebrae, omitting a positioning frame, according to another embodiment of the disclosure.

FIG. 5 is a three-dimensional diagram illustrating a first support body according to an embodiment of the disclosure.

FIG. 6 is a three-dimensional diagram illustrating a second support body according to an embodiment of the disclosure.

FIG. 7 is a three-dimensional diagram illustrating a first support body according to another embodiment of the disclosure.

FIG. 8 is a three-dimensional diagram illustrating a positioning frame according to an embodiment of the disclosure.

FIG. 9 is an assembly diagram illustrating a pushing component, a first sliding column, and a second sliding column according to an embodiment of the disclosure.

FIG. 10 is an assembly diagram illustrating a pushing component, a first sliding column, a second sliding column, and a driving rod according to an embodiment of the disclosure.

FIG. 11 is an assembly diagram illustrating a driving rod according to an embodiment of the disclosure.

FIG. 12 is a three-dimensional diagram illustrating a snap ring according to an embodiment of the disclosure.

### REFERENCE NUMERALS IN THE DRAWINGS

intervertebral implant 100;
first support body 1; first support main body 11; first support part 12; second support part 13; first inclined slot 14; second inclined slot 15; first positioning slot 16; first protrusion 17; first connecting hole 18; bone implanting window 19;
second support body 2; second support main body 21; third support part 22; fourth support part 23; third inclined slot 24; fourth inclined slot 25; second positioning slot 26; second protrusion 27; second connecting hole 28;
driving component 3; pushing component 31; accommodation part 311; through hole 312; first sliding column 32; first column body 321; first limiting protrusion 322; second sliding column 33; second column body 331; second limiting protrusion 332; driving rod 34; mating part 341; connecting part 342; end part 343; snap ring 35;
positioning frame 4; frame body 41; mating hole 411; bone implanting hole 412; holding slot 413; avoidance slot 414; first guiding protrusion 42; second guiding protrusion 43;
first connecting component 51; second connecting component 52.

### DETAILED DESCRIPTION

Description will be made in detail below to embodiments of the disclosure. Examples of embodiments are illustrated in the accompanying drawings. Embodiments described below with reference to the accompanying drawings are exemplary, which are intended to explain the disclosure and do not be understood a limitation of the disclosure.

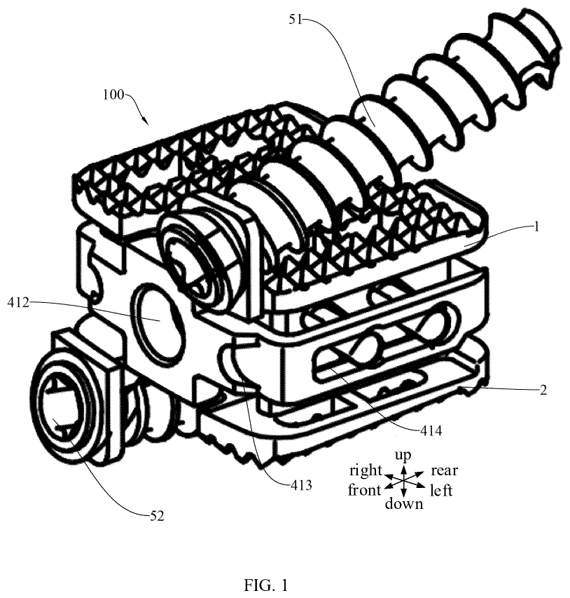

Description will be made below to an intervertebral implant 100 for distracting a cervical vertebrae and an intervertebral implantation system in embodiments of the disclosure with reference to FIG. 1 to FIG. 12.

The intervertebral implant 100 for distracting the cervical vertebrae in embodiments of the disclosure includes a driving component 3, a first support body 1, and a second support body 2. The intervertebral implant 100 has an expanded state and a collapsed state. The first support body 1 and the second support body 2 are separately provided along a height direction of the intervertebral implant 100 to form a bone accommodation cavity. The first support body 1 is provided with a first inclined slot 14 that is inclined relative to the height direction of the intervertebral implant 100, and the first inclined slot 14 is provided with a notch open towards the second support body 2. The driving component 3 is provided between the first support body 1 and the second support body 2 in the height direction of the intervertebral implant 100. The driving component 3 includes a pushing component 31 and a first sliding column 32 connected with each other. The first sliding column 32 cooperatively slides in the first inclined slot 14, and the pushing component 31 moves along a direction perpendicular to the height direction of the intervertebral implant 100 and perpendicular to an extending direction of the first sliding column 32, to realize transition between the collapsed state and the expanded state of the intervertebral implant 100 by the pushing component 31 driving the first sliding column 32 to move along an inclined direction of the first inclined slot 14.

In other words, the pushing component 31 moves along the direction perpendicular to the height direction of the intervertebral implant 100 and perpendicular to the extending direction of the first sliding column 32. For example, the pushing component 31 may move back and forth along the length direction or a width direction of the intervertebral implant 100.

It may be understood that the first support body 1 and the second support body 2 spread outward or close inward relative to each other, in which outward spreading of the intervertebral implant 100 may correspond to an increase in an angle between the first support body 1 and the second support body 2. The first support body 1 and the second support body 2 are sequentially provided along the height direction of the intervertebral implant 100. For example, as illustrated in FIG. 1, the first support body 1, the driving component 3, and the second support body 2 are sequentially provided from an up-to-down direction. The depth direction of the first inclined slot 14 is specifically at an angle relative to the height direction of the intervertebral implant 100.

With the intervertebral implant 100 for distracting the cervical vertebrae in embodiments of the disclosure, through the first inclined slot 14 of the first support body 1 and the first sliding column 32 of the driving component 3, the movement of the driving component 31 along the direction perpendicular to the height direction of the intervertebral implant 100 and perpendicular to the extending direction of the first sliding column 32 is transitioned into the movement of the first sliding column 32 along the height direction of the intervertebral implant 100, such that the transition between the collapsed state and the expanded state of the intervertebral implant 100 is realized, with an advantage of a simple structure. In addition, as the first sliding column 32 cooperates with the first inclined slot 14, the pushing component 31 also moves along the height direction of the intervertebral implant 100, such that a volume occupied by the pushing component 31 between the first support body 1 and the second support body 2 does not change with the movement of the pushing component 31 while raising upper and lower heights of the first support body 1 and the second support body 2, which correspondingly enhances a space of the bone accommodation cavity during the process of the transition between the collapsed state and the expanded state of the intervertebral implant 100, enhances the implantable bone graft materials and improves the bone fusion effect of distracting the intervertebral implant 100. Such way has the advantage of improving the stability and fusion performance of the intervertebral implant. In addition, the intervertebral implant may be adequately distracted, which enhances an effect of restoring a physiological curvature of the cervical vertebrae.

Thus, the intervertebral implant 100 for distracting the cervical vertebrae in embodiments of the disclosure has advantages such a simple structure, a good bone implant effect, and a high rate of bone fusion.

For example, as illustrated in FIG. 1, the first sliding column 32 extends along the width direction of the intervertebral implant 100, then the pushing component 31 moves along the length direction of the intervertebral implant 100. The first inclined slot 14 is inclined from front to rear along the up-to-down direction.

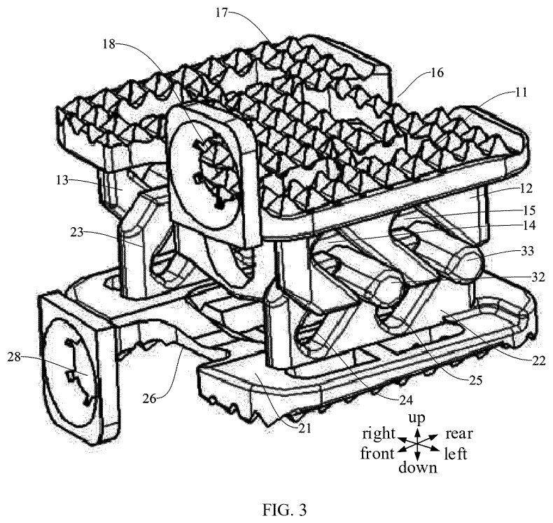

As illustrated in FIG. 3 and FIG. 5, the first support body 1 includes a first support main body 11, a first support part 12 (e.g., the left side illustrated in FIG. 3), and a second support part 13 (e.g., the right side illustrated in FIG. 3). The first support part 12 and the second support part 13 are symmetrically provided on two sides of the first support main body 11 along the width direction of the intervertebral implant 100. The first support part 12 and the second support part 13 extend from the first support main body 11 to a direction close to the second support body 2 along the height direction of the intervertebral implant 100. Each of the first support part 12 and the second support part 13 (the first support part 12 and the second support part 13 being symmetrically provided along a width direction of the first support body 11) is provided with a first inclined slot 14. There are at least two first sliding columns 32, and the two first sliding columns 32 are provided in the first inclined slot 14. The pushing component 31 is movably provided between the first support body 1 and the second support body 2 along the length direction of the intervertebral implant 100. It may be understood that each of the first support part 12 and the second support part 13 is provided with the first inclined slot 14. The first inclined slot 14 of the first support part 12 and the first inclined slot 14 of the second support part 13 are symmetrically provided along the width direction of the intervertebral implant 100. The at least two first sliding columns 32 are symmetrically provided along the width direction of the intervertebral implant on both sides of the pushing component 31. The first sliding column 32 cooperatively slides in the first inclined slot 14 along the depth direction of the first inclined slot 14.

With the intervertebral implant 100 for distracting the cervical vertebrae in embodiments of the disclosure, the first support part 12 and the second support part 13 extend along the height direction of the intervertebral implant 100, and the first sliding column 32 is correspondingly provided in the first inclined slot 14 of the first support part 12 and the second support part 13, thus enhancing the space of the bone accommodation cavity in the first support body 1, the second support body 2, and the driving component 3. Such way provides the advantages of further enhancing the stability and fusion performance of the intervertebral implant 100.

Further, providing the first support part 12 and the second support part 13 on both sides of the first support main body 11 and correspondingly providing the first sliding column 32 on both sides of the pushing component 31 have the advantage of a excellent structural stability.

As illustrated in FIG. 3 to FIG. 7, each of the first support part 12 and the second support part 13 are symmetrically provided with a second inclined slot 15 along the width direction of the intervertebral implant 100. The driving component 3 includes at least two second sliding columns 33, the second sliding column 33 is correspondingly provided within the second inclined slot 15. The first inclined slot 14 and the second inclined slot 15 are sequentially provided along the length direction (e.g., a front-to-rear direction illustrated in FIG. 1 to FIG. 3) of the intervertebral implant 100. It may be understood that the second inclined slot 15 of the first support part 12 and the second inclined slot 15 of the second support part 13 are symmetrically provided along the width direction of the intervertebral implant 100. It may be understood that an inclined direction of the first inclined slot 14 is the same as an inclined direction of the second inclined slot 15.

With the intervertebral implant 100 for distracting a cervical vertebrae in embodiments of the disclosure, each of the first support part 12 and the second support part 13 is provided with the second inclined slot 15, and the first inclined slot 14 and the second inclined slot 15 are provided sequentially along the length direction of the intervertebral implant 100, such that synchronous adjustment of the height along the length direction of the intervertebral implant 100 is performed during the transition between the collapsed state and the expanded state of the intervertebral implant 100, thus enhancing the structural stability and adjustment synchronization of the intervertebral implant 100.

As illustrated in FIG. 7, the first support main body 11 has a front end and a rear end along the length direction of the intervertebral implant 100, the first inclined slot 14 is provided adjacent to the front end, the second inclined slot 15 is provided adjacent to the rear end, and an inclination angle of the first inclined slot 14 is smaller than an inclination angle of the second inclined slot 15, to enable the pushing component 31 to move along the length direction of the intervertebral implant 100 to change a relative distance and a relative angle between the first support body 1 and the second support body 2. It may be understood that the inclined direction of the first inclined slot 14 is the same as the inclined direction of the second inclined slot 15, but inclination angles of the two inclined slots are different. The inclination angle of the first inclined slot 14 is an angle between the depth direction of the first inclined slot 14 and the height direction of the intervertebral implant 100. The inclination angle of the second inclined slot 15 is an angle between the depth direction of the second inclined slot 15 and the height direction of the intervertebral implant 100.

With the intervertebral implant 100 for distracting the cervical vertebrae in embodiments of the disclosure, the inclination angle of the first inclined slot 14 is configured to be smaller than the inclination angle of the second inclined slot 15, a distance that the first sliding column 32 moves up on the intervertebral implant 100 is greater than a distance that the second sliding column 33 moves up on the intervertebral implant 100 when the pushing component 31 moves a same distance. Thus, the angle between the first support body 1 and the second support body 2 is adjusted, such that the intervertebral implant 100 may adapt to different patients and the applicability of the intervertebral implant 100 is improved.

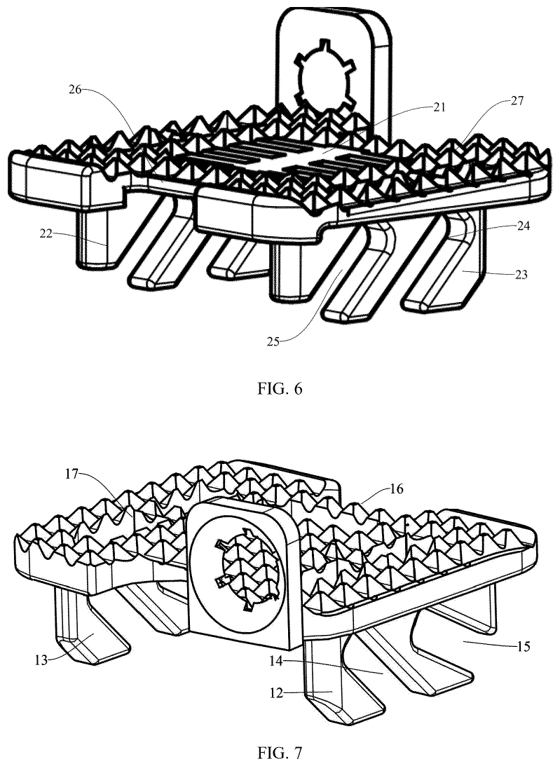

As illustrated in FIG. 3 and FIG. 6, the second support body 2 is provided with a third inclined slot 24 open toward a side of the first support body 1. An inclined direction of the third inclined slot 24 is opposite to the inclined direction of the first inclined slot 14 (the inclination angles may be the same or different). The third inclined slot 24 intersects with the first inclined slot 14, and the first sliding column 321 corresponds to a depth direction of the third inclined slot 24 and is movably provided within the third inclined slot 24, to enable the first support body 1 and the second support body 2 to approach or separate from each other. It may be understood that the first sliding column 32 not only moves along the inclined direction of the first inclined slot 14, and the first sliding column 32 also corresponds to the depth direction of the third inclined slot 24 and movably cooperates within the third inclined slot 24.

With the intervertebral implant 100 for distracting the cervical vertebrae in embodiments of the disclosure, the first sliding slot 14 provided on the first support body 1 intersects with the third sliding slot 24 provided on the second support body 2, and the first sliding columns 32 may simultaneously slide in both the first inclined slot 14 and the third inclined slot 24, such that the first support body 1 and the second support body 2 may approach or separate from each other, thus providing the advantage of improving the height adjustment efficiency of the intervertebral implant 100

As illustrated in FIG. 3, the first inclined slot 14 is inclined from front to rear along an up-to-down direction, the third inclined slot 24 is inclined from rear to front along the up-to-down direction. The first inclined slot 14 is inclined at 45° from front to rear along the up-to-down direction, the third inclined slot 24 is inclined at 45° from rear to front along the up-to-down direction. The inclination angle of the first inclined slot 14 is less than or equal to the inclination angle of the second inclined slot 24.

As illustrated in FIG. 3 and FIG. 5, the second support body 2 includes a second support main body 21, a third support part 22, and a fourth support part 23. The third support part 22 and the fourth support part 23 are symmetrically provided on two sides of the second support main body 21 along the width direction of the intervertebral implant 100. The third support part 22 and the fourth support part 23 extend from the second support main body 21 to a direction close to the first support body 1. Each of the third support part 22 and the fourth support part 23 is provided with the third inclined slot 24, and the first sliding column 32 corresponds to a depth direction of the third inclined slot 24 and is movably provided within the third inclined slot 24.

It may be understood that each of the third support part 22 and the fourth support part 23 is provided with the third inclined slot 24. The first inclined slot 14 of the third support part 22 and the third inclined slot 24 of the fourth support part 23 are symmetrically provided along the width direction of the intervertebral implant 100. There are at least two first sliding columns 32, the at least two first sliding columns 32 are symmetrically provided along the width direction of the intervertebral implant on both sides of the pushing component 31, and the first sliding columns 32 cooperatively slide in the third sloping slots 24.

With the intervertebral implant 100 for distracting the cervical vertebrae in embodiments of the disclosure, the third support part 22 and the fourth support part 23 extend along the height direction of the intervertebral implant 100, to enable the second support main body 21, the third support part 22, and the fourth support part 23 to form an accommodation space, thus enhancing the space for accommodating the bone implant material among the third support part 22, the fourth support part 23, and the driving component 3, and providing the advantage of further improving the stability and fusion performance of the intervertebral implant 100.

Further, the third inclined slot 24 of the third support part 22 and the third inclined slot 24 of the fourth support part 23 are provided on both sides of the second support main body 21, and the first sliding column 32 is correspondingly provided on both sides of the pushing component 31, thus providing the advantage of enhancing the force balance of the second support main body 21.

As illustrated in FIG. 3 to FIG. 7, the third inclined slot 24 and the first inclined slot 14 are symmetrically provided on a preset plane perpendicular to the height direction of the intervertebral implant 100.

With the intervertebral implant 100 for distracting the cervical vertebrae in embodiments of the disclosure, the third inclined slot 24 and the first inclined slot 14 are symmetrically provided on a preset plane perpendicular to the height direction of the intervertebral implant 100, such that there is no relative offset of the first support body 1 and the second support body 2 along an anterior-posterior direction of the intervertebral implant 100 when the first support body 1 and the second support body 2 approach or separate from each other. Thus, the structural stability of the intervertebral implant 100 is further improved.

As illustrated in FIG. 3 and FIG. 6, the second support body 2 includes a second support main body 21, a third support part 22, and a fourth support part 23. The third support part 22 and the fourth support part 23 are symmetrically provided on two ends of the second support main body 21 along the width direction of the intervertebral implant 100. Each of the third support part 22 and the fourth support part 23 is provided with a third inclined slot 24 and a fourth inclined slot 25. The first sliding column 32 corresponds to a depth direction of the first inclined slot 14 and is movably provided within the third inclined slot 24. The second sliding column 33 corresponds to a depth direction of the fourth inclined slot 25 and is movably provided within the fourth inclined slot 25. The third inclined slot 24 and the first inclined slot 14 are symmetrically provided on the preset plane perpendicular to the height direction of the intervertebral implant 100. The fourth inclined slot 25 and the second inclined slot 15 are symmetrically provided along the preset plane, to enable the first support body 1 and the second support body 2 to approach or separate from each other.

It may be understood that each of the first support part 12 and the second support part 13 is provided with the first inclined slot 14 and the second inclined slot 15. Each of the third support part 22 and the fourth support part 23 is provided with the third inclined slot 24 and the fourth inclined slot 25.

With the intervertebral implant 100 for distracting the cervical vertebrae in embodiments of the disclosure, each of the first support part 12 and the second support part 13 is provided with the first inclined slot 14 and the second inclined slot 15, and each of the third support part 22 and the fourth support part 23 provided with the third inclined slot 24 and the fourth inclined slot 25, such that synchronous adjustment along the length direction of the intervertebral implant 100 may be performed during the transition between the collapsed state and the expanded state of the intervertebral implant 100, thus providing the advantage of further enhancing the structural stability and adjustment synchronization of the intervertebral implant 100.

In addition, the inclination angle of the first inclined slot 14 is smaller than the inclination angle of the second inclined slot 15, such that the heights and angles of the first support body 1 and the second support body 2 may be changed while the pushing component 31 moves along the length direction of the intervertebral implant 100. When the pushing component 31 moves a same distance, the distance that the first sliding column 32 moves up on the intervertebral implant 100 is greater that the distance that the second sliding column 33 moves up on the intervertebral implant 100. Thus, the angle between the first support body 1 and the second support body 2 is adjusted, such that the intervertebral implant 100 may adapt to different patients and improve the applicability of the intervertebral implant 100. For example, as illustrated in FIG. 3, the first inclined slot 14 and the second inclined slot 15 are inclined from front to rear along the up-to-down direction, the inclination angle of the first inclined slot 14 is 45°, and the inclination angle of the second inclined slot 15 is 50°. The third inclined slot 24 is inclined from front to rear along the up-to-down direction, the inclination angle of the third inclined slot 24 is 45°, and the inclination angle of the fourth inclined slot 25 is 50°.

In addition, the third inclined slot 24 and the first inclined slot 14 are symmetrically provided on the preset plane perpendicular to the height direction of the intervertebral implant 100. The fourth inclined slot 25 and the second inclined slot 15 are symmetrically provided along the preset plane. The first support body 1 and the second support body 2 may be approached or separated from each other, and the first support body 1 and the second support body 2 do not move relative to each other along the anterior-posterior direction of the intervertebral implant 100. Thus, the structural stability of the intervertebral implant 100 is further improved.

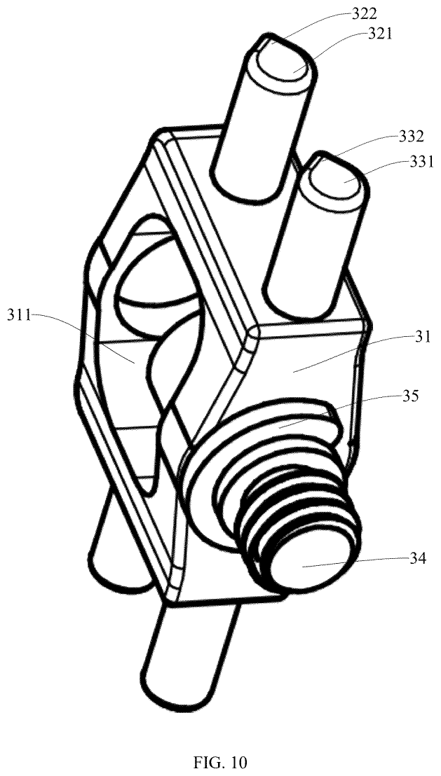

As illustrated in FIG. 9 and FIG. 10, the first sliding column 32 includes a first column body 321 and a first limiting protrusion 322. The first limiting protrusion 322 has a first sliding surface, and the first sliding surface is in contact with a slot wall surface of the first inclined slot 14. The second sliding column 33 includes a second column body 331 and a second limiting protrusion 332, the second limiting protrusion 332 has a second sliding surface, and the second sliding surface is in contact with a slot wall surface of the second inclined slot 15.

With the intervertebral implant 100 for distracting the cervical vertebrae in embodiments of the disclosure, the first sliding column 32 includes the first column body 321 and the first limiting protrusion 322, which may increase a contact area of the first sliding column 32 with the first inclined slot 14. Correspondingly, the second sliding column 33 includes the second column body 331 and the second limiting protrusion 332, which may increase a contact area of the second sliding column 33 with the second inclined slot 15.

As illustrated in FIG. 9 and FIG. 10, the first limiting protrusion 322 further includes a third sliding surface. The third sliding surface is in contact with a slot wall surface of the third inclined slot 24. The first sliding surface and the third sliding surface are symmetrically provided on the preset plane perpendicular to the height direction of the intervertebral implant 100. Each of the first sliding surface and the third sliding surface is tangent to a peripheral wall of the first column body, to enable the first sliding column 32 to form a cross-section in a water-droplet shape.

With the intervertebral implant 100 for distracting the cervical vertebrae in embodiments of the disclosure, by symmetrically providing the first sliding surface and the second sliding surface on the preset plane perpendicular to the height direction of the intervertebral implant 100, and providing each of the first sliding surface and the third sliding surface to be tangent to the peripheral wall of the first column body, the contact area of the first sliding column 32 with the first inclined slot 14 may be further increased.

In some embodiments, a cross-section of the first sliding column 32 is in a water-droplet shape. Thus, not only the contact area of the first sliding column 32 with the first inclined slot 14 is increased, but also the second sliding column 33 is prevented from disengaging from the second inclined slot 15 and the fourth inclined slot 25.

The second limiting protrusion 232 further includes a fourth sliding surface. The fourth sliding surface is in contact with a slot wall surface of the fourth inclined slot 25. The second sliding surface and the fourth sliding surface are symmetrically provided on the preset plane perpendicular to the height direction of the intervertebral implant 100, and each of the second sliding surface and the fourth sliding surface is tangent to a peripheral wall of the second column body, to enable the second sliding column 33 to form a cross-section in a water-droplet shape.

In some embodiments, a cross-section of the second sliding column 33 is in a water-droplet shape. Thus, not only the contact area of the second sliding column 33 with the second inclined slot 15 is increased, but also the second sliding column 33 is prevented from disengaging from the second inclined slot 15 and the fourth inclined slot 25.

In some embodiments, an angle between the first sliding surface and the third sliding surface is 90°≤α≤180°.

In some embodiments, an angle between the second sliding surface and the fourth sliding surface is 90°≤α≤180°.

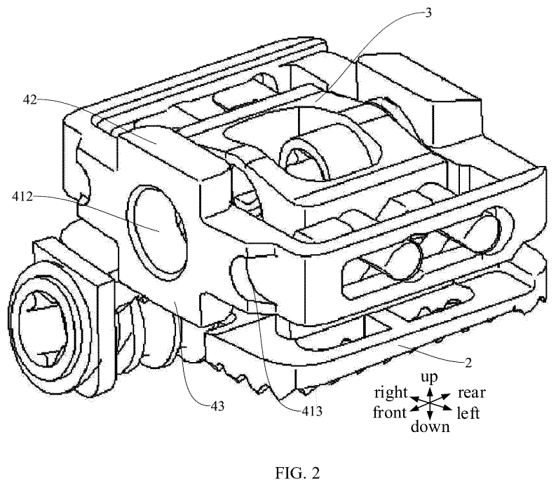

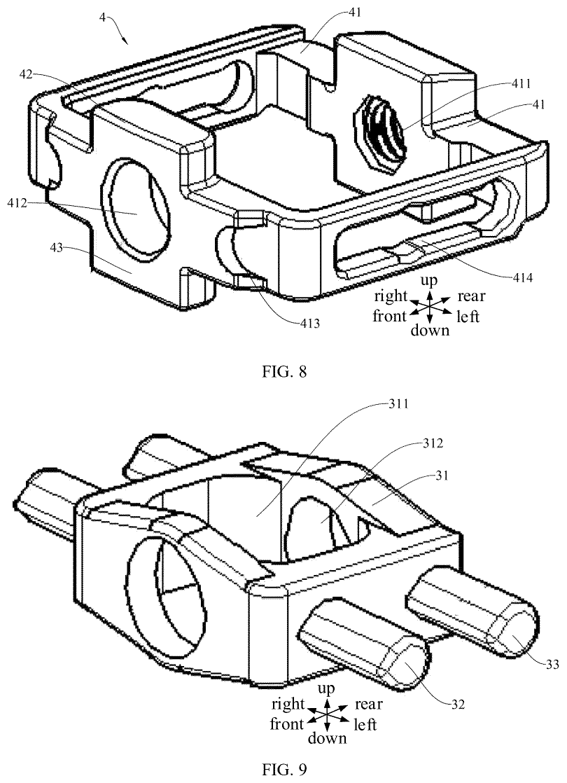

As illustrated in FIG. 2 and FIG. 8, the intervertebral implant 100 for distracting the cervical vertebrae further includes a positioning frame 4. The positioning frame 4 is sleeved outside the first support body 1 and the second support body 2, to avoid or reduce a displacement of the first support body 1 and the second support body 2 in a direction perpendicular to the height direction of the intervertebral implant 100.

With the intervertebral implant 100 for distracting the cervical vertebrae in embodiments of the disclosure, by providing the positioning frame 4 to cooperate with each of the first support body 1 and the second support body 2, the positions of the first support body 1 and the second support body 2 along the width direction and the height direction of the intervertebral implant 100 are limited, thus providing the advantage of further enhancing the structural stability of the intervertebral implant 100.

As illustrated in FIG. 2 and FIG. 8, the positioning frame 4 includes a frame body 41 and a first guiding protrusion 42. The first guiding protrusion 42 is provided on one side of the frame body 41 and extended toward one side close to the first support body 1. The first support body 1 is provided with a first positioning slot 16, and the first guiding protrusion 42 is movably provided within the first positioning slot along the height direction of the intervertebral implant 100.

With the intervertebral implant 100 for distracting the cervical vertebrae in embodiments of the disclosure, the first guiding protrusion 42 of the positioning frame 4 is guidedly cooperated with the first positioning slot 16 of the first support body 1 along the height direction of the intervertebral implant 100, thus providing the advantage of further enhancing the structural stability of the intervertebral implant 100

As illustrated in FIG. 2 and FIG. 8, the positioning frame 4 further includes a second guiding protrusion 43. The second guiding protrusion 43 is provided on one side of the frame body 41 and extended toward one side close to the second support body 2. The second support body 2 is provided with a second positioning slot 26, and the second guiding protrusion 43 is movably provided within the second positioning slot along the height direction of the intervertebral implant 100.

With the intervertebral implant 100 for distracting the cervical vertebrae in embodiments of the disclosure, i with the second guiding protrusion 43 of the positioning frame 4 is guidedly cooperated with the second positioning slot 26 of the second support body 2 along the height direction of the intervertebral implant 100, thus providing the advantage of further enhancing the structural stability of the intervertebral implant 100.

In some embodiments, there may be multiple first guiding protrusions 42, in which two of the first guiding protrusions 42 are provided relative to each other on the frame body 41 along the length direction or the width direction of the intervertebral implant 100, thus providing the advantage of further enhancing the structural stability of the intervertebral implant 100. For example, as illustrated in FIG. 8, there are two first guiding protrusions 42, and the two first guiding protrusions 42 are provided relative to each other on the frame body 41 along the length direction of the intervertebral implant 100.

In some embodiments, there may be multiple second guiding protrusions 43, in which two of the second guiding protrusions 43 are provided relative to each other on the frame body 41 along the length direction or the width direction of the intervertebral implant 100, thus providing the advantage of further enhancing the structural stability of the intervertebral implant 100. For example, as illustrated in FIG. 8, there are two second guiding protrusions 43, and the two second guiding protrusions 43 are provided relative to each other on the frame body 41 along the length direction of the intervertebral implant 100.

In some embodiments, the first guiding protrusion 42 and the second guiding protrusion 43 are provided opposite to each other on the frame body 41 along the height direction of the intervertebral implant 100.

As illustrated in FIG. 9 to FIG. 12, the driving component 3 further includes a driving rod 34, the driving rod 34 includes a mating part 341, a connecting part 342, and an end part 343 sequentially connected. A step is formed between the connecting part 342 and the end part 343. The frame body 41 is provided with a mating hole 411, the pushing component 31 is provided with a through hole, the mating part 341 is inserted into the mating hole 411, the connecting part 342 is inserted into the through hole 312, and the driving component 3 abuts against the step.

With the intervertebral implant 100 for distracting the cervical vertebrae in embodiments of the disclosure, the driving rod 34 may cooperate with the movement of the driving component 31, so as to realize the transition between the collapsed state and the expanded state of the intervertebral implant 100, thus providing the advantage of ease of operation.

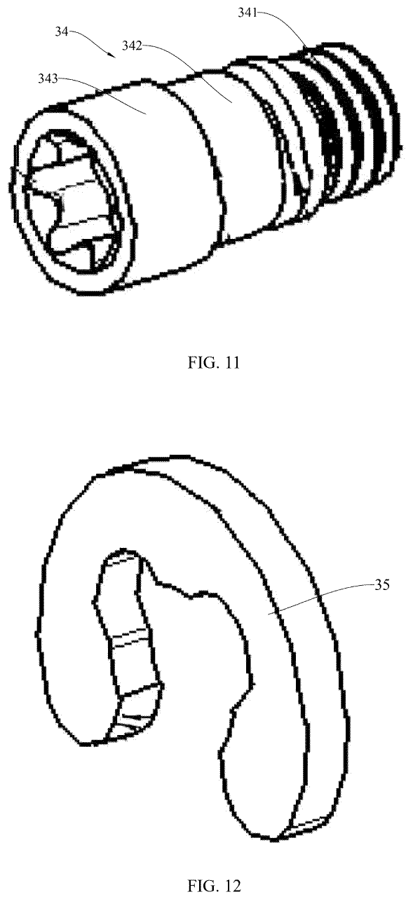

In some embodiments, as illustrated in FIG. 11, the mating hole 411 is a threaded hole, and the mating part 341 is a threaded segment.

In some embodiments, the end part 343 of the drive rod 34 is provided with one of a plum groove, a cross groove, or a monogram groove.

With the intervertebral implant 100 for distracting the cervical vertebrae in embodiments of the disclosure, the driving component 3 may move along the length direction of the intervertebral implant 100 through rotation of the driving component 3, and the pushing component 31 is driven to move along the length direction of the intervertebral implant 100 by further inserting the connecting part 342 into the through hole 312 and abutting the driving component 3 against the step, to enable transition between the collapsed state and the expanded state of the intervertebral implant 100, thus providing the advantage of improving the precision and controllability of adjustments.

In some embodiments, as illustrated in FIG. 10 to FIG. 12, the snap ring 35 is additionally sleeved on the driving rod 34 between the pushing component 31 and the frame body 41, to prevent the driving rod 34 from disengaging from the threaded hole. It may be understood that when the driving rod 34 is about to disengage from the threaded hole, the snap ring 35 mates with the driving rod 34 to block further movement of the driving rod 34 in the direction of disengagement from the threaded hole, thus improving the operational reliability of the intervertebral implant 100.

As illustrated in FIG. 9 and FIG. 10, the frame body 41 has a front side and a rear side along the length direction of the intervertebral implant 100. The front side is provided with a bone implanting hole 412 for filling bone implant material into the bone accommodation cavity. Thus, when the intervertebral implant 100 is transitioned to the expanded state from the collapsed state, the bone accommodation cavity is filled with the bone implant material, thereby providing the advantage of good bone implant effect and high rate of bone fusion.

In some embodiments, the mating hole 411 is provided on the rear side of the frame body 41, the bone implanting hole 412 is provided on the front side of the frame body 41, and the through hole 312, the mating hole 411, and the bone implanting hole 412 of the pushing component 31 are provided in a same straight line.

During implantation of the intervertebral implant 100, the implanter sequentially passes through the bone implanting hole 412 and the through hole 312, and connects to the driving rod 34, then the driving rod 34 is driven to rotate. Thus, the driving rod 34 moves along the anterior-posterior direction of the intervertebral implant 100 as the driving rod 34 rotates. As illustrated in FIG. 3 and FIG. 8, the front side of the frame body 41 is further provided with a holding slot 413 or a holding protrusion for implanting a wrench to clamp the frame body 41.

With the intervertebral implant 100 for distracting the cervical vertebrae in embodiments of the disclosure, the holding slot 413 or the holding protrusion is provided on the front side of the frame body 41, thus facilitating the implanter to position the positioning frame 4. Thus, when the driving component 3 moves along the anterior-posterior direction of the intervertebral implant 100, the relative movement between the first support body 1 and the second support body 2 may be avoided. Thus, the structural reliability of the intervertebral implant 100 is improved.

As illustrated in FIG. 3 and FIG. 8, each of two sides of the frame body 41 along the width direction of the intervertebral implant 100 is provided with an avoidance slot.

With the intervertebral implant 100 for distracting the cervical vertebrae in embodiments of the disclosure, by providing the avoidance slots 414 on the two sides of the frame body 41 along the width direction of the intervertebral implant 100, the interference of the first sliding column 32 with the frame body 41 may be avoided.

As illustrated in FIG. 9 and FIG. 10, the pushing component 31 is a hollow accommodating component 311.

With the intervertebral implant 100 for distracting the cervical vertebrae in embodiments of the disclosure, by providing the pushing component 31 as the hollow accommodating component 311, the space of the bone accommodation cavity may be further enhanced. Thus, an amount of implantable bone graft materials is enhanced, and the bone fusion effect of the distracted intervertebral implant is improved.

As illustrated in FIG. 3 to FIG. 7, a side of the first support body 1 far away from the second support body 2 is provided with multiple first protrusions 17. A side of the second support body 2 far away from the first support body 1 is provided with multiple second protrusions 27.

With the intervertebral implant 100 for distracting the cervical vertebrae in embodiments of the disclosure, by providing the multiple first protrusions 17 on the side of the first support body 1 far away from the second support body 2, the fusion effect between the first support body 1 and a vertebra may be enhanced. Accordingly, by providing the multiple second protrusions 27 on the side of the second support body 2 far away from the first support body 1, the fusion effect between the second support body 2 and the vertebra may also be enhanced.

In some embodiments, the inclination angles of the multiple first protrusions 17 may differ from each other, and similarly, the inclination angles of the multiple second protrusions 27 may also differ from each other. Thus, protrusions with different inclination angles may achieve more thorough fusion with corresponding vertebrae and better alignment with the intervertebral physiological angle, thereby improving the surgical outcomes of the intervertebral implant 100.

As illustrated in FIG. 5 and FIG. 7, the side of the first support body 1 far away from the second support body 2 is a convex curved surface. Thus, the conformity between the first support body 1 and the vertebra (a mating surface of the vertebra being a concave curved surface) is enhanced.

In some embodiments, the side of the first support body 1 far away from the second support body 2 is an arcuate surface.

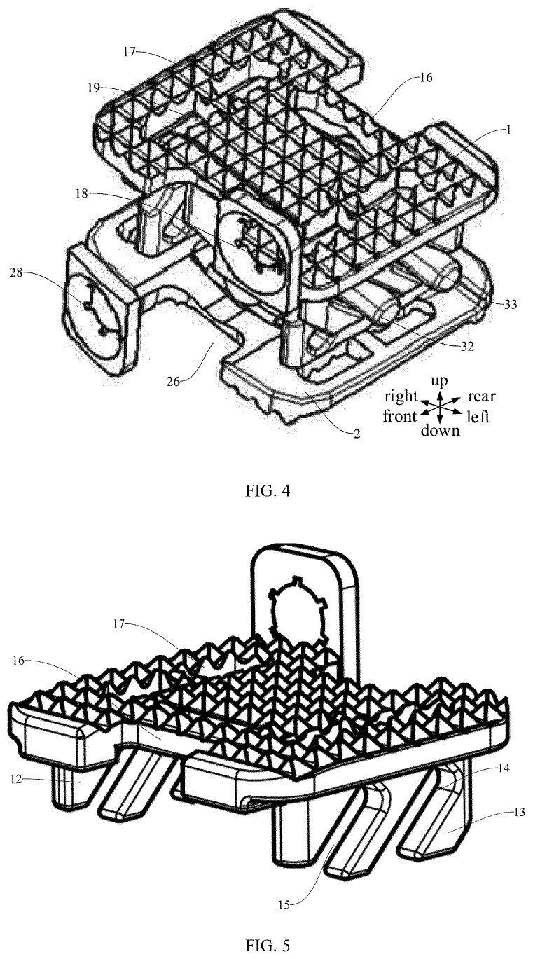

As illustrated in FIG. 2 and FIG. 4, a thickness of the second support body 2 decreases from one end (e.g., the front end illustrated in FIG. 2) of the second support body 2 to the other end (e.g., the rear end illustrated in FIG. 2) of the second support body 2 along the length direction of the intervertebral implant 100.

In some embodiments, the thickness of the second support body 2 gradually decreases from front to rear along the length direction of the intervertebral implant 100.

At least one of the first support body 1 and the second support body 2 is a porous structure (not illustrated). In other words, the first support body 1 is provided with the porous structure, or the second support body 2 is provided with the porous structure. Alternatively, each of the first support body 1 and the second support body 2 is provided with the porous structure. Thus, the fusion effect between the first support body 1, the second support body 2, and the upper and lower vertebrae may improved, enhancing the post-implantation stability of the intervertebral implant 100.

As illustrated in FIG. 4, at least one of the first support body 1 and the second support body 2 is provided with a bone implanting window 19. Thus, the fusion effect between the first support body 1, the second support body 2, and the upper and lower vertebrae may improved, enhancing the post-implantation stability of the intervertebral implant 100.

In some embodiments, the first support body 1 and the second support body 2 may be made of materials such as titanium alloy, tantalum, niobium-zirconium alloy, etc.

In some embodiments, at least one of the first support body 1 and the second support body 2 is a 3D-printed component. For example, both the first support body 1 and the second support body 2 may be 3D-printed components, allowing the first support body 1 and the second support body 2 to be printed in different models and specifications based on actual requirements of different patients, and shortening the manufacturing process of the intervertebral implant 100 and reducing the production cost of the intervertebral implant 100.

As illustrated in FIG. 1, FIG. 3, and FIG. 4, the intervertebral implant for distracting the cervical vertebrae also includes a first connecting component 51. The first support body 1 is provided with a first connecting hole 18 for insertion of the first connecting component 51, to enable the first connecting component 51 to connect with the vertebra. Thus, the stability of the connection between the intervertebral implant 100 and the vertebra may be further improved.

As illustrated in FIG. 1, FIG. 3, and FIG. 4, the intervertebral implant for distracting the cervical vertebrae also includes a second connecting component 52. The second support body 2 is provided with a second connecting hole 28 for insertion of the second connecting component 52, to enable the second connecting component 52 to connect with the vertebra. Thus, the stability of the connection between the intervertebral implant 100 and the vertebra may be further improved.

In some embodiments, the first connecting component 51 and the second connecting component 52 may be screws, and both the first connecting hole 18 and the second connecting hole 28 are threaded holes.

The intervertebral implant system in embodiments of the disclosure includes an implanter and the intervertebral implant 100 for distracting the cervical vertebrae according to any one of the above. The implanter is connected to the pushing component 31 and configured to adjust a position of the pushing component 31, to realize the transition between the collapsed state and the expanded state of the intervertebral implant 100 by the pushing component 31 driving the first sliding column 32 to move along the inclined direction of the first inclined slot 14. Thus, the intervertebral implant 100 for distracting the cervical vertebrae in embodiments of the disclosure has the advantage of a good bone implant effect and a high rate of bone fusion.

The application of the intervertebral implant for distracting the cervical vertebrae is provided in embodiments of the disclosure. The intervertebral implant is applied to the cervical vertebra, a thoracic vertebra, or a lumbar vertebra.

In the description of the disclosure, it is to be understood that, terms such as “center”, “longitudinal”, “lateral”, “length”, “width”, “thickness”, “over”, “below”, “front”, “back”, “left”, “right”, “vertical”, “horizontal”, “up”, “down”, “in”, “out”, “clockwise”, “anti-clockwise”, “axial”, “radial” and “circumference” refer to the directions and location relations which are the directions and location relations illustrated in the drawings, and for describing the disclosure and for describing in simple, and which are not intended to indicate or imply that the device or the elements are disposed to locate at the specific directions or are structured and performed in the specific directions, which could not to be understood to the limitation of the disclosure.

In addition, terms such as “first” and “second” are used herein for purposes of description and are not intended to indicate or imply relative importance or significance, or implicitly indicate the number of technical features indicated. Furthermore, the feature defined with “first” and “second” may include at least one this feature distinctly or implicitly. In the description of the disclosure, “a plurality of” or “multiple” means at least two, e.g., two, three, etc., unless specified otherwise.

In the disclosure, unless specified or limited otherwise, the terms “mounted,” “connected,” “coupled” and “fixed” are understood broadly, such as fixed, detachable mountings, or integrated connections, and can be mechanical or electrical mountings or communicable with each other, connections and couplings, and also can be direct and via media indirect mountings, connections, and couplings, and further can be inner mountings, connections and couplings of two components or interaction relations between two components, unless specified otherwise. The above terms can be understood by those skilled in the art according to the detail embodiment of the disclosure.

In the disclosure, unless specified or limited otherwise, the first feature is “on” or “under” the second feature refers to the first feature and the second feature can be in a direct contact or via media indirect contact. And, the first feature “on”, “above”, “over” the second feature may refer to that the first feature is right over the second feature or is diagonal above the second feature, or just refer to the horizontal height of the first feature is higher than the horizontal height of the second feature. The first feature “below” or “under” the second feature may refer to that the first feature is right over the second feature or is diagonal under the second feature, or just refer to the horizontal height of the first feature is lower than the horizontal height of the second feature.

In the description of the disclosure, reference throughout this specification to “an embodiment,” “some embodiments,” “an example,” “a specific example,” or “some examples,” means that a detailed feature, structure, material, or characteristic described in connection with the embodiment or example is included in at least one embodiment or example of the disclosure. Thus, the appearances of the phrases in various places throughout this specification are not necessarily referring to the same embodiment or example of the disclosure. Furthermore, the particular features, structures, materials, or characteristics may be combined in any suitable manner in one or more embodiments or examples. Without a contradiction, the different embodiments or examples and the features of different embodiments or examples can be combined by those skilled in the art.

Although embodiments of the disclosure have been illustrated and described, it should be understood that the above embodiments are exemplary and cannot be construed to limit the disclosure, and changes, modifications, alternatives, and variations can be made in the embodiments without departing from scope of the disclosure.

1. An intervertebral implant for distracting a cervical vertebrae, wherein the intervertebral implant has an expanded state and a collapsed state, and the intervertebral implant comprises:
a first support body and a second support body, the first support body and the second support body being separately provided along a height direction of the intervertebral implant to form a bone accommodation cavity, the first support body being provided with a first inclined slot that is inclined relative to the height direction of the intervertebral implant, and the first inclined slot being provided with a notch open towards the second support body; and
a driving component, provided between the first support body and the second support body in the height direction of the intervertebral implant, the driving component comprising a pushing component and a first sliding column connected with each other, the first sliding column cooperatively sliding in the first inclined slot, and the pushing component moving along the height direction of the intervertebral implant and perpendicular to an extending direction of the first sliding column, to realize transition between the collapsed state and the expanded state of the intervertebral implant by the driving component driving the first sliding column to move along an inclined direction of the first inclined slot.

2. The intervertebral implant of claim 1, wherein the first support body comprises a first support main body, a first support part, and a second support part, the first support part and the second support part are symmetrically provided on two sides of the first support main body along a width direction of the intervertebral implant, the first support part and the second support part extend from the first support main body to a direction close the second support body along the height direction of the intervertebral implant, each of the first support part and the second support part is provided with the first inclined slot, there are at least two first sliding columns, the first sliding column cooperatively slides in the first inclined slot along a depth direction of the first inclined slot, and the pushing component is movably provided between the first support body and the second support body along a length direction of the intervertebral implant.

3. The intervertebral implant of claim 2, wherein each of the first support part and the second support part is provided with a second inclined slot, the driving component comprises at least two second sliding columns, the at least two second sliding columns are correspondingly provided within the second inclined slots, and the first inclined slot and the second inclined slot are sequentially provided along the length direction of the intervertebral implant.

4. The intervertebral implant of claim 3, wherein the first support main body has a front end and a rear end along the length direction of the intervertebral implant, the first inclined slot is provided adjacent to the front end, the second inclined slot is provided adjacent to the rear end, and an inclination angle of the first inclined slot is smaller than an inclination angle of the second inclined slot, to enable the pushing component to move along the length direction of the intervertebral implant to change a height and an angle between the first support body and the second support body.

5. The intervertebral implant of claim 2, wherein the second support body is provided with a third inclined slot open toward a side of the first support body, the third inclined slot intersects with the first inclined slot, and the first sliding column corresponds to a depth direction of the third inclined slot and is movably provided within the third inclined slot, to enable the first support body and the second support body to approach or separate from each other.

6. The intervertebral implant of claim 5, wherein the second support body comprises a second support main body, a third support part, and a fourth support part, the third support part and the fourth support part are symmetrically provided on two sides of the second support main body along the width direction of the intervertebral implant, the third support part and the fourth support part extend from the second support main body to a direction close to the first support body, each of the third support part and the fourth support part is provided with the third inclined slot, and the first sliding column corresponds to a depth direction of the third inclined slot and is movably provided within the third inclined slot; and/or
the third inclined slot and the first inclined slot are symmetrically provided on a preset plane perpendicular to the height direction of the intervertebral implant.

7. The intervertebral implant of claim 3, wherein the second support body comprises a second support main body, a third support part, and a fourth support part, the third support part and the fourth support part are symmetrically provided on two ends of the second support main body along the width direction of the intervertebral implant, each of the third support part and the fourth support part is provided with a third inclined slot and a fourth inclined slot, the first sliding column corresponds to a depth direction of the first inclined slot and is movably provided within the third inclined slot, the second sliding column corresponds to a depth direction of the fourth inclined slot and is movably provided within the fourth inclined slot, the third inclined slot and the first inclined slot are symmetrically provided on a preset plane perpendicular to the height direction of the intervertebral implant, and the fourth inclined slot and the second inclined slot are symmetrically provided along the preset plane, to enable the first support body and the second support body to approach or separate from each other.

8. The intervertebral implant of claim 7, wherein the first inclined slot is inclined at 45° from front to rear along an up-to-down direction, the third inclined slot is inclined at 45° from rear to front along the up-to-down direction, and an inclination angle of the first inclined slot is less than or equal to an inclination angle of the second inclined slot.

9. The intervertebral implant of claim 7, wherein the first sliding column comprises a first column body and a first limiting protrusion, the first limiting protrusion has a first sliding surface, and the first sliding surface is in contact with a slot wall surface of the first inclined slot; and/or
the second sliding column comprises a second column body and a second limiting protrusion, the second limiting protrusion has a second sliding surface, and the second sliding surface is in contact with a slot wall surface of the second inclined slot.

10. The intervertebral implant of claim 9, wherein the first limiting protrusion further comprises a third sliding surface, the third sliding surface is in contact with a slot wall surface of the third inclined slot, the first sliding surface and the second sliding surface are symmetrically provided on the preset plane perpendicular to the height direction of the intervertebral implant, and each of the first sliding surface and the third sliding surface is tangent to a peripheral wall of the first column body; and/or,
the second limiting protrusion further comprises a fourth sliding surface, the fourth sliding surface is in contact with a slot wall surface of the fourth inclined slot, the second sliding surface and the fourth sliding surface are symmetrically provided on the preset plane perpendicular to the height direction of the intervertebral implant, and each of the second sliding surface and the fourth sliding surface is tangent to a peripheral wall of the second column body.

11. The intervertebral implant of claim 10, wherein a cross-section of the first sliding column is in a water-droplet shape;
a cross-section of the second sliding column is in a water-droplet shape;
an angle α between the first sliding surface and the third sliding surface is 90°≤α≥180°; and/or,
an angle α between the second sliding surface and the fourth sliding surface is 90°≤α≤180°.

12. The intervertebral implant of claim 1, further comprising a positioning frame, the positioning frame being sleeved outside the first support body and the second support body to limit a displacement of the first support body and the second support body in a direction perpendicular to the height direction of the intervertebral implant.

13. The intervertebral implant of claim 12, wherein the positioning frame comprises at least one of a frame body and a first guiding protrusion; or a second guiding protrusion, wherein the first guiding protrusion is provided on one side of the frame body and extended toward one side close to the first support body, the first support body is provided with a first positioning slot, and the first guiding protrusion is movably provided within the first positioning slot along the height direction of the intervertebral implant;
wherein the second guiding protrusion is provided on one side of the frame body and extended toward one side close to the second support body, the second support body is provided with a second positioning slot, and the second guiding protrusion is movably provided within the second positioning slot along the height direction of the intervertebral implant.

14. The intervertebral implant of claim 13, wherein the driving component further comprises a driving rod, the driving rod comprises a mating part, a connecting part, and an end part sequentially connected, a step is formed between the connecting part and the end part, the frame body is provided with a mating hole, the pushing component is provided with a through hole, the mating part is inserted into the mating hole, the connecting part is inserted into the through hole, and the driving component abuts against the step.

15. The intervertebral implant of claim 13, wherein the frame body has a front side and a rear side along the length direction of the intervertebral implant, and the front side is provided with a bone implanting hole for filling bone implant material into the bone accommodation cavity;
the front side of the frame body is further provided with a holding slot or a holding protrusion for implanting a wrench to clamp the frame body; and/or
each of two sides of the frame body along the width direction of the intervertebral implant is provided with an avoidance slot.

16. The intervertebral implant of claim 1 wherein the pushing component is a hollow accommodating component.

17. The intervertebral implant of claim wherein a side of the first support body far away from the second support body is provided with a plurality of first protrusions;
the side of the first support body far away from the second support body is a convex curved surface (arcuate surface);
a side of the second support body far away from the first support body is provided with a plurality of second protrusions;
a thickness of the second support body decreases from one end of the second support body to the other end of the second support body along the length direction of the intervertebral implant;
at least one of the first support body and the second support body is a porous structure; and/or
at least one of the first support body and the second support body is provided with a bone implanting window.

18. The intervertebral implant of claim 1, further comprising at least one of a first connecting component; or second connecting component, wherein the first support body being provided with a first connecting hole for insertion of the first connecting component, so as to the first connecting component to connect with a vertebra;
wherein the second support body being provided with a second connecting hole for insertion of the second connecting component, so as to the second connecting component to connect with the vertebra.

19. An intervertebral implantation system, comprising an implanter and an intervertebral implant for distracting a cervical vertebrae, the implanter being connected to a pushing component and configured to adjust a position of the pushing component, to realize transition between a collapsed state and an expanded state of the intervertebral implant by the pushing component driving a first sliding column to move along an inclined direction of a first inclined slot.

20. An application of an intervertebral implant for distracting a cervical vertebrae, wherein the intervertebral implant is applied to a cervical vertebra, a thoracic vertebra, or a lumbar vertebra.
